(19) United States
(12) Patent Application Publication
(10) Pub. No.: US 2024/0216153 A1
(43) Pub. Date: Jul. 4, 2024

(54) ENDOPROSTHESIS FOR ENHANCED SIDE BRANCH ACCESS

(71) Applicant: BOSTON SCIENTIFIC SCIMED, INC., Maple Grove, MN (US)

(72) Inventors: Martyn G. Folan, Galway (IE); Gary Gilmartin, Foxford (IE); Louis McNern, Donegal (IE); Daniel Tuck, Galway (IE); Jonathan Root, Townsend, MA (US)

(73) Assignee: BOSTON SCIENTIFIC SCIMED, INC., Maple Grove, MN (US)

(21) Appl. No.: 18/403,488

(22) Filed: Jan. 3, 2024

Related U.S. Application Data
(60) Provisional application No. 63/437,030, filed on Jan. 4, 2023.

(51) Int. Cl. A61F 2/856; A61F 2/90

(52) U.S. Cl. CPC A61F 2/856; A61F 2/90; A61F 2210/0076; A61F 2220/0008; A61F 2240/001

(57) ABSTRACT

An endoprosthesis includes an elongated tubular member including a filament forming circumferential rows having a plurality of knitted loops and intermediate rungs extending between adjacent knitted loops. The tubular member includes at least one window and may include a plurality of anti-migration loops adjacent the at least one window and configured to shift between first and second positions when unconstrained. Making the endoprosthesis includes forming the plurality of knitted loops and intermediate rungs in a first row of loops and rungs, extending the filament to form a second row of loops and rungs interwoven with the first row, and forming the at least one window without cutting the filament. Placing the endoprosthesis includes positioning the endoprosthesis within a body lumen adjacent a branch lumen, expanding the tubular member to span the branch lumen, and shifting at least some anti-migration loops toward the second position and into the branch lumen.

### CROSS-REFERENCE TO RELATED APPLICATIONS

This application claims the benefit of U.S. Provisional Patent Application Ser. No. 63/437,030 filed on Jan. 4, 2023, the disclosure of which is incorporated herein by reference.

### TECHNICAL FIELD

The present disclosure pertains to medical devices, systems, and methods for manufacturing and/or using medical devices and/or systems. More particularly, the present disclosure pertains to an endoprosthesis or stent for implantation in a body lumen, and associated methods.

### BACKGROUND

A wide variety of medical devices have been developed for surgical and/or intravascular use. Implantable medical devices (e.g., endoprostheses, stents, etc.) may be designed to treat a variety of medical conditions in the body. For example, some endoprostheses or stents may be designed to radially expand and support a body lumen and/or provide a fluid pathway for digested material, blood, or other fluid to flow therethrough following a medical procedure. Some medical devices may include radially or self-expanding stents which may be implanted transluminally via a variety of medical device delivery systems. These stents may be implanted in a variety of body lumens such as coronary or peripheral arteries, the esophageal tract, gastrointestinal tract (including the intestine, stomach, and/or colon), tracheobronchial tract, urinary tract, biliary tract, vascular system, etc.

In some instances, it may be desirable to design stents to include sufficient flexibility while maintaining sufficient radial force to open the body lumen at the treatment site. In some instances, the radial force may aid in anchoring the stent and preventing migration within the lumen. In some instances, different stent configurations may provide different deliverability, flexibility, radial force/strength, and/or anchoring characteristics. For example, in some configurations, knitted stents are considered to possess superior flexibility and/or radial strength characteristics. However, knitted stents have certain limitations with respect to re-constrainment and/or repositioning. In some instances, a knitted stent may be the most appropriate or desirable type of stent for a particular lumen or use—for example, in gastrointestinal and/or tracheobronchial anatomy. In some situations, placement of a stent may cause undesirable consequences. For example, treatment of a body lumen at and/or adjacent a branch lumen may result in the stent obstructing the branch lumen, which may reduce or block fluid flow into or out of the branch lumen. One example of such placement may be within the biliary tree.

Of the known medical devices and methods, each has certain advantages and disadvantages. There is an ongoing need to provide alternative medical devices as well as alternative methods for manufacturing and using medical devices. Examples of endoprostheses or stents including features intended to overcome issues related to side branch access and/or drainage are disclosed herein.

### SUMMARY

In one example, an endoprosthesis may comprise an elongated tubular member comprising a filament forming circumferential rows disposed about a central longitudinal axis, the circumferential rows having a plurality of knitted loops and intermediate rungs extending between adjacent knitted loops. The filament may extend continuously from a first end of the elongated tubular member to a second end of the elongated tubular member. The elongated tubular member may include at least one window formed through a side wall of the elongated tubular member having a size equal to or greater than two knitted loops of the plurality of knitted loops.

In addition or alternatively to any example disclosed herein, intermediate rungs extend across the at least one window.

In addition or alternatively to any example disclosed herein, the at least one window is devoid of intermediate rungs.

In addition or alternatively to any example disclosed herein, at a perimeter of the at least one window the filament shifts from one circumferential row to an adjacent circumferential row and reverses circumferential direction around the central longitudinal axis of the elongated tubular member.

In addition or alternatively to any example disclosed herein, the elongated tubular member includes a plurality of anti-migration loops disposed adjacent the at least one window, the plurality of anti-migration loops being configured to shift between a first position extending generally parallel to the side wall and a second position extending radially outward from the side wall. The plurality of anti-migration loops is self-biased toward the second position.

In addition or alternatively to any example disclosed herein, in the first position, at least one first anti-migration loop of the plurality of anti-migration loops extends toward the first end of the elongated tubular member and at least one second anti-migration loop of the plurality of anti-migration loops extends toward the second end of the elongated tubular member.

In addition or alternatively to any example disclosed herein, the at least one first anti-migration loop overlaps the at least one second anti-migration loop in the first position.

In addition or alternatively to any example disclosed herein, the plurality of anti-migration loops extends across the at least one window in the first position.

In addition or alternatively to any example disclosed herein, the plurality of anti-migration loops includes a polymeric covering disposed thereon.

In addition or alternatively to any example disclosed herein, each anti-migration loop of the plurality of anti-migration loops has an outer perimeter greater than an outer perimeter of each knitted loop of the plurality of knitted loops.

In addition or alternatively to any example disclosed herein, the elongated tubular member includes a polymeric covering disposed thereon between the first end and the second end.

In addition or alternatively to any example disclosed herein, the at least one window is devoid of the polymeric covering.

In addition or alternatively to any example disclosed herein, a method of making an endoprosthesis comprising an elongated tubular member may comprise: knitting a filament around a central longitudinal axis to form a plurality of knitted loops and intermediate rungs extending between adjacent knitted loops in a first row of loops and rungs; extending the filament around the central longitudinal axis to form a second row of loops and rungs interwoven with the first row; and forming at least one window through a side wall of the elongated tubular member having a size equal to or greater than two knitted loops of the plurality of knitted loops without cutting the filament.

In addition or alternatively to any example disclosed herein, forming at least one window through the side wall of the elongated tubular member includes shifting the filament from its current row of loops and rungs to an adjacent row of loops and rungs, forming at least one knitted loop in the adjacent row of loops and rungs, and shifting the filament back to the current row of loops and rungs to form at least one additional knitted loop.

In addition or alternatively to any example disclosed herein, forming at least one window through the side wall of the elongated tubular member includes shifting the filament from its current row of loops and rungs to a first subsequent row of loops and rungs, and forming the first subsequent row of loops and rungs interwoven with the current row of loops and rungs.

In addition or alternatively to any example disclosed herein, a method of placing an endoprosthesis in a body lumen having a branch lumen may comprise: positioning an endoprosthesis within the body lumen adjacent the branch lumen, wherein the endoprosthesis comprises an elongated tubular member comprising a filament forming a plurality of knitted loops and intermediate rungs extending between adjacent knitted loops, wherein the elongated tubular member includes at least one window formed through a side wall of the elongated tubular member having a size equal to or greater than two knitted loops of the plurality of knitted loops, and a plurality of anti-migration loops disposed adjacent the at least one window, the plurality of anti-migration loops being configured to shift between a first position extending generally parallel to the side wall and a second position extending radially outward from the side wall; expanding the elongated tubular member against a wall of the body lumen such that the elongated tubular member spans an opening into the branch lumen; and shifting at least some of the plurality of anti-migration loops from the first position toward the second position, wherein the at least some of the plurality of anti-migration loops shifted toward the second position extend into the opening of the branch lumen.

In addition or alternatively to any example disclosed herein, after shifting the at least some of the plurality of anti-migration loops from the first position toward the second position, all remaining loops of the plurality of anti-migration loops are trapped between the side wall of the elongated tubular member and the wall of the body lumen in the first position.

In addition or alternatively to any example disclosed herein, the plurality of anti-migration loops at least partially covers the at least one window in the first position.

In addition or alternatively to any example disclosed herein, the endoprosthesis is positioned within the body lumen adjacent the branch lumen such that the at least one window overlaps the opening of the branch lumen.

In addition or alternatively to any example disclosed herein, the plurality of anti-migration loops is formed from the filament forming the elongated tubular member.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates selected aspects of an endoprosthesis in a delivery configuration according to the disclosure;

FIG. 2 illustrates selected aspects of an endoprosthesis in a deployed configuration according to the disclosure;

FIG. 3 illustrates selected aspects of an endoprosthesis according to the disclosure;

FIG. 4-5 illustrate selected aspects of an endoprosthesis having a plurality of anti-migration loops according to the disclosure;

FIG. 6 illustrates selected aspects of the endoprosthesis of FIGS. 4-5 positioned in a body lumen adjacent a branch lumen;

FIG. 7 is a flat pattern view illustrating selected aspects of a portion of an endoprosthesis according to the disclosure;

FIG. 8 is a flat pattern view illustrating selected aspects of a portion of an endoprosthesis according to the disclosure;

FIG. 9 illustrates selected aspects of the endoprosthesis of FIG. 8 positioned in a body lumen adjacent a branch lumen;

FIG. 10 is a flat pattern view illustrating selected aspects of a portion of an endoprosthesis according to the disclosure;

FIG. 11 is a flat pattern view illustrating selected aspects of a portion of an endoprosthesis according to the disclosure along with a side view schematically illustrating aspects of the flat pattern view;

FIG. 12 is a flat pattern view illustrating selected aspects of a portion of an endoprosthesis according to the disclosure;

FIG. 13 is a flat pattern view illustrating selected aspects of a portion of the endoprosthesis of FIG. 12 according to the disclosure along with a side view schematically illustrating aspects of the flat pattern view;

FIG. 14 illustrates selected aspects of the endoprosthesis of FIGS. 12-13 positioned in a body lumen adjacent a branch lumen; and

FIG. 15 is a flat pattern view illustrating selected aspects of a portion of an endoprosthesis according to the disclosure.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all of the components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail herein.

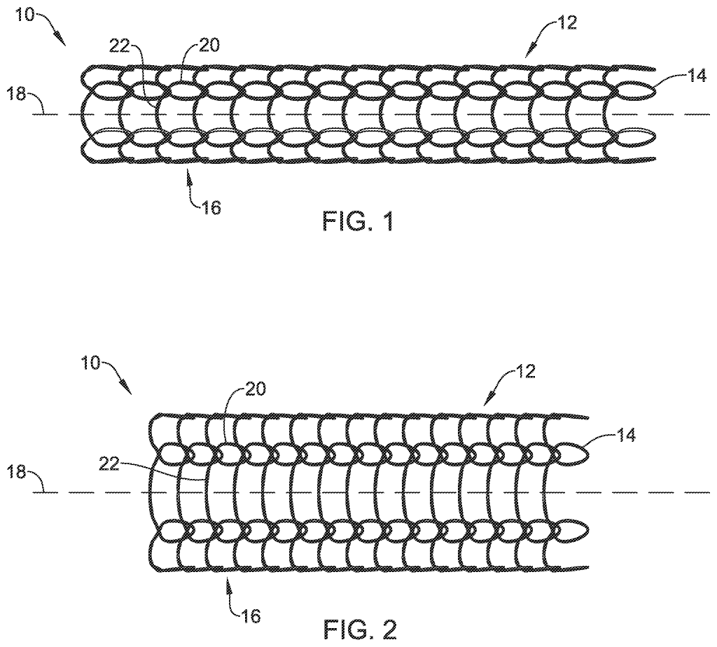

FIGS. 1-2 illustrate selected elements and/or features of an endoprosthesis 10 according to the disclosure. For reference, the terms endoprosthesis and stent may be used interchangeably herein. In some embodiments, the endoprosthesis 10 may comprise an elongated tubular member 12 comprising a filament 14 forming circumferential rows 16 disposed about a central longitudinal axis 18.

In some embodiments, the filament 14 and/or the elongated tubular member 12 may comprise one or more interwoven filaments. In some embodiments, the filament 14 and/or the elongated tubular member 12 may comprise a plurality of interwoven filaments. In some embodiments, the endoprosthesis 10 and/or the elongated tubular member 12 may have a knitted structure formed and/or fabricated from one or more filaments (or a plurality of filaments) interwoven with each other along the length of the endoprosthesis 10 and/or the elongated tubular member 12.

In at least some embodiments, the filament 14 may extend continuously from a first end of the elongated tubular member 12 to a second end of the elongated tubular member 12. Accordingly, in some embodiments, the elongated tubular member 12 may be formed from a single filament or only one filament. In some embodiments, the endoprosthesis 10 and/or the elongated tubular member 12 may have a knitted structure formed and/or fabricated by knitting a single filament extending the entire length of the endoprosthesis 10 and/or the elongated tubular member 12. Some suitable but non-limiting examples of materials that may be used to form the elongated tubular member 12 and/or the filament 14 including but not limited to polymeric materials, metallic materials, composites, and the like, are described below.

In some embodiments, the circumferential rows 16 may include a plurality of knitted loops 20 and intermediate rungs 22 extending between adjacent knitted loops of and/or within the circumferential rows 16. In at least some embodiments, the plurality of knitted loops 20 may be closed loops. In some alternative configurations, the plurality of knitted loops 20 may be open loops or partially open loops. In some embodiments, the plurality of knitted loops 20 may be a mixture of open loops, partially open loops, and/or closed loops. The circumferential rows 16 may extend circumferentially around the central longitudinal axis 18. In some embodiments, the filament 14 and/or the circumferential rows 16 may define a side wall of the elongated tubular member 12 extending from the first end of the elongated tubular member 12 to the second end of the elongated tubular member 12.

In some embodiments, the endoprosthesis 10 and/or the elongated tubular member 12 may be configured to expand from a delivery configuration (e.g., FIG. 1) toward and/or to a deployed configuration (e.g., FIG. 2). In some embodiments, the endoprosthesis 10 and/or the elongated tubular member 12 may be configured to self-expand from the delivery configuration toward and/or to the deployed configuration. In some embodiments, the endoprosthesis 10 and/or the elongated tubular member 12 may be self-biased toward the deployed configuration. In some alternative configurations, the endoprosthesis 10 and/or the elongated tubular member 12 may be mechanically expandable, balloon expandable, etc. from the delivery configuration toward and/or to the deployed configuration.

In some embodiments, the delivery configuration may be a radially collapsed configuration and/or an axially elongated configuration. In some embodiments, the deployed configuration may be a radially expanded configuration and/or an axially foreshortened configuration. In some embodiments, the endoprosthesis 10 and/or the elongated tubular member 12 may be advanced and/or delivered to a treatment site in the delivery configuration using a delivery device. In some embodiments, the delivery device may constrain the endoprosthesis 10 and/or the elongated tubular member 12 in the delivery configuration. Upon deployment at the treatment site, the endoprosthesis 10 and/or the elongated tubular member 12 may expand radially outward and/or may axially foreshorten toward and/or to the deployed configuration. In some embodiments, the endoprosthesis 10 and/or the elongated tubular member 12 may minimally foreshorten while radially expanding, which may be desirable in certain applications.

While not expressly shown, in some embodiments, the endoprosthesis 10 and/or the elongated tubular member 12 may include a first flared region adjacent the first end and/or a second flared region adjacent the second end of the endoprosthesis 10 and/or the elongated tubular member 12. In some embodiments, the first flared region and/or the second flared region may have an enlarged outer extent and/or an enlarged outer diameter relative to a body of the endoprosthesis 10 and/or the elongated tubular member 12 disposed between the first end and the second end. Other configurations are also contemplated.

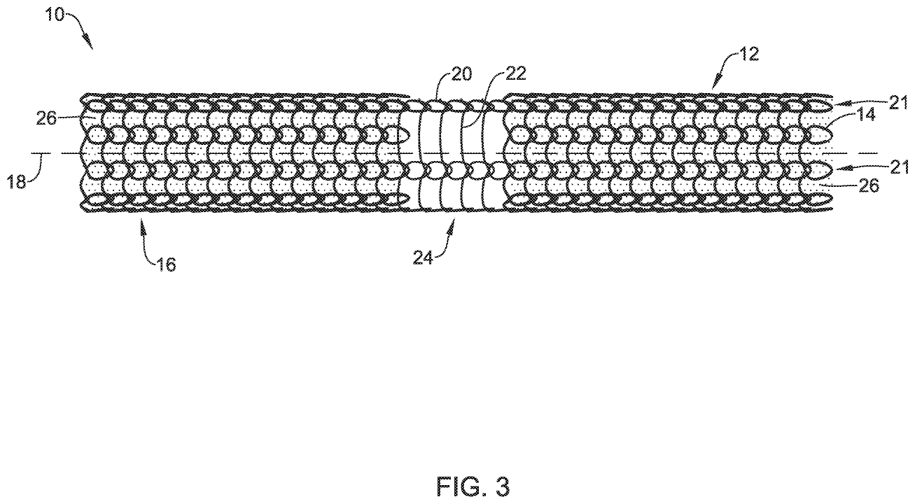

In some embodiments, the elongated tubular member 12 may include at least one window 24 formed through the side wall of the elongated tubular member 12, as seen in FIG. 3 for example. In at least some embodiments, the at least one window 24 may have a perimeter defined by columns of knitted loops 21 along axially extending sides of the at least one window 24 and by circumferential rows 16 along circumferentially extending sides of the at least one window 24. The perimeter of the at least one window 24 may be a closed perimeter. In some embodiments, the at least one window 24 may have a size equal to or greater than two knitted loops of the plurality of knitted loops 20. In some embodiments, the at least one window 24 may be devoid of knitted loops within the perimeter of the at least one window 24. In the non-limiting example illustrated in FIG. 3, each window of the at least one window 24 has a size equal to or greater than five knitted loops of the plurality of knitted loops 20 (e.g., each window is devoid of five knitted loops within the perimeter thereof). Other configurations and/or sizes of windows in accordance with the disclosure are also contemplated.

In some embodiments, the at least one window 24 may include a plurality of windows arranged around a circumference of the elongated tubular member 12 with an axially oriented column of knitted loops extending between circumferentially adjacent windows and/or circumferentially consecutive windows of the plurality of windows. In some embodiments, the at least one window 24 may include a plurality of windows arranged around the circumference of the elongated tubular member 12 with a plurality of axially oriented columns of knitted loops 21 extending between circumferentially adjacent windows and/or circumferentially consecutive windows of the plurality of windows. Other configurations, including combinations thereof, are also contemplated.

In some embodiments, intermediate rungs 22 extend across the at least one window 24, as seen in FIG. 3. In some embodiments, intermediate rungs 22 extend circumferentially across the at least one window 24. In some embodiments, at least two intermediate rungs 22 extend across the at least one window 24. In some embodiments, the intermediate rungs 22 extending across the at least one window 24 have a circumferential length greater than intermediate rungs 22 that do not extend across the at least one window 24. In some embodiments, intermediate rungs 22 disposed within the perimeter of the at least one window have a circumferential length greater than intermediate rungs 22 defining the perimeter of the at least one window 24 and/or disposed outside of the perimeter of the at least one window 24. For example, by removing some knitted loops from within the perimeter of the at least one window 24, intermediate rungs 22 extending across the at least one window 24 are longer than those defining the perimeter and/or disposed outside of the perimeter of the at least one window 24.

In some embodiments, the elongated tubular member 12 may include a polymeric covering 26 disposed thereon between the first end and the second end. In some embodiments, the at least one window 24 may be devoid of the polymeric covering 26. In some embodiments, the elongated tubular member 12 may include a first polymeric covering portion adjacent the first end and a second polymeric covering portion adjacent the second end, wherein the first polymeric covering portion is spaced apart from the second polymeric covering portion. In some embodiments, the polymeric covering 26 may be disposed on the plurality of knitted loops 20 extending between adjacent windows of the at least one window 24. In some embodiments, the polymeric covering 26 may extend from the first end to the second end. In some embodiments, the polymeric covering 26 may extend from the first end to the second end and/or the polymeric covering 26 may extend over the at least one window 24. In some embodiments, the polymeric covering 26 may extend from the first end to the second end except for the at least one window 24. Some suitable but non-limiting examples of materials that may be used to form the polymeric covering 26 are described below.

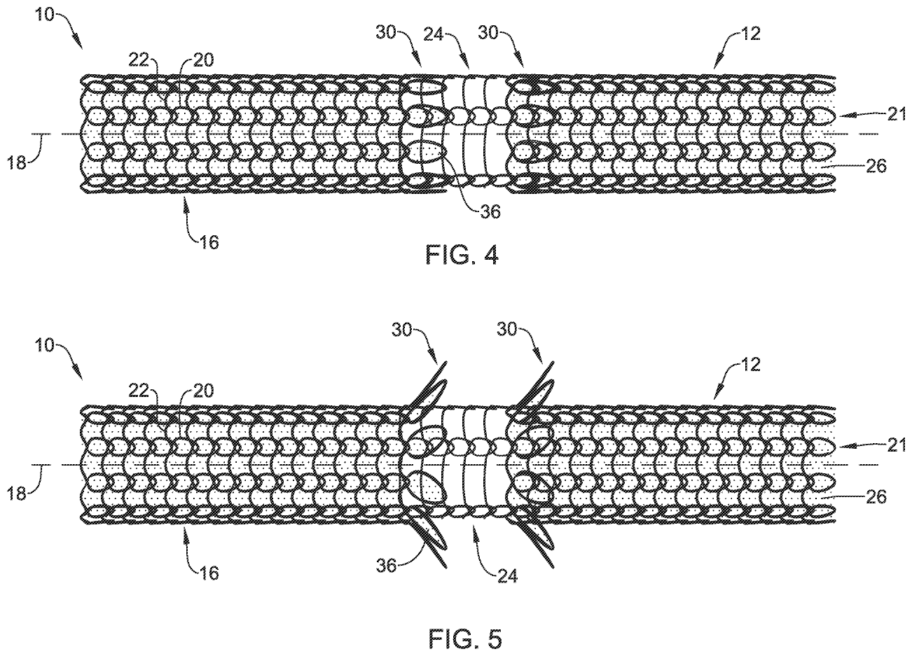

In some embodiments, the endoprosthesis 10 and/or the elongated tubular member 12 may comprise and/or include a plurality of anti-migration loops 30 disposed adjacent the at least one window 24, as seen in FIGS. 4-5 for example. The plurality of anti-migration loops 30 may be configured to shift between a first position (e.g., FIG. 4) extending generally parallel to the side wall and/or the central longitudinal axis 18 and a second position (e.g., FIG. 5) extending radially outward from the side wall and/or the central longitudinal axis 18. In at least some embodiments, the plurality of anti-migration loops 30 may be self-biased toward the second position.

In at least some embodiments, the plurality of anti-migration loops 30 may be formed from the filament 14. In some embodiments, the plurality of anti-migration loops 30 may be disposed around the circumference of the elongated tubular member 12. In some embodiments, the plurality of anti-migration loops 30 may be disposed completely around the circumference of the elongated tubular member 12 and/or around the entire circumference of the elongated tubular member 12.

In some embodiments, the plurality of anti-migration loops 30 may be arranged in one or more rows of anti-migration loops. In some embodiments, the one or more rows of anti-migration loops may extend circumferentially around the elongated tubular member 12 and/or the central longitudinal axis 18. In some embodiments, the filament 14 may be doubled up around the circumference of the elongated tubular member 12 and/or within one or more circumferential rows 16 to form the plurality of anti-migration loops 30 and/or the one or more rows of anti-migration loops. In some embodiments, the filament 14 may be interwoven with an existing circumferential row 16 when forming the plurality of anti-migration loops 30 and/or the one or more rows of anti-migration loops. In some embodiments, the plurality of anti-migration loops 30 and/or the one or more rows of anti-migration loops may axially overlap an existing circumferential row 16 when forming the plurality of anti-migration loops 30 and/or the one or more rows of anti-migration loops. In some alternative embodiments, the plurality of anti-migration loops 30 and/or the one or more rows of anti-migration loops may be formed from a separate filament from the filament 14 forming the elongated tubular member 12. In those embodiments, the separate filament forming the plurality of anti-migration loops 30 and/or the one or more rows of anti-migration loops may be interwoven with the filament 14, the circumferential rows 16, and/or the elongated tubular member 12.

In some embodiments, the plurality of anti-migration loops 30 may extend radially outward from the side wall and/or the central longitudinal axis 18 at an oblique angle relative to the side wall and/or the central longitudinal axis 18 in the second position and/or when unconstrained. In some embodiments, the plurality of anti-migration loops 30 may extend radially outward from the side wall and/or the central longitudinal axis 18 at an acute angle relative to the side wall and/or the central longitudinal axis 18 in the second position and/or when unconstrained. In some embodiments, the plurality of anti-migration loops 30 may extend radially outward from the side wall and/or the central longitudinal axis 18 at a right angle relative to the side wall and/or the central longitudinal axis 18 in the second position and/or when unconstrained. In some embodiments, the plurality of anti-migration loops 30 may extend radially outward from the side wall and/or the central longitudinal axis 18 at an obtuse angle relative to the side wall and/or the central longitudinal axis 18 in the second position and/or when unconstrained.

In some embodiments, the plurality of anti-migration loops 30 may extend radially outward from the side wall and/or the central longitudinal axis 18 at an angle of about 30 degrees, about 45 degrees, about 60 degrees, about 75 degrees, about 90 degrees, about 105 degrees, about 120 degrees, about 135 degrees, about 150 degrees, etc. relative to the side wall and/or the central longitudinal axis 18 in the second position and/or when unconstrained. In some embodiments, some anti-migration loops of the plurality of anti-migration loops 30 may extend radially outward from the side wall and/or the central longitudinal axis 18 at different angles from each other relative to the side wall and/or the central longitudinal axis 18 in the second position and/or when unconstrained. Other configurations are also contemplated.

In some embodiments, the plurality of anti-migration loops 30 may extend across the at least one window 24 in the first position. In some embodiments, the plurality of anti-migration loops 30 may extend partially across the at least one window 24 in the first position. In some embodiments, the plurality of anti-migration loops 30 may extend completely across the at least one window 24 in the first position. In some embodiments, the plurality of anti-migration loops 30 may cover at least a portion of the at least one window 24 in the first position. In some embodiments, the plurality of anti-migration loops 30 may cover at least 50% of the at least one window 24 in the first position. In some embodiments, the plurality of anti-migration loops 30 may cover at least 60% of the at least one window 24 in the first position. In some embodiments, the plurality of anti-migration loops 30 may cover at least 70% of the at least one window 24 in the first position. In some embodiments, the plurality of anti-migration loops 30 may cover at least 80% of the at least one window 24 in the first position. In some embodiments, the plurality of anti-migration loops 30 may cover at least 90% of the at least one window 24 in the first position. In some embodiments, the plurality of anti-migration loops 30 may completely cover the at least one window 24 in the first position. In some embodiments, the plurality of anti-migration loops 30 may overlap each other in the first position. In some embodiments, the plurality of anti-migration loops 30 may be positioned next to and/or adjacent each other without overlapping each other in the first position.

In some embodiments, the plurality of anti-migration loops 30 may include a polymeric covering 36 disposed thereon. In some embodiments, the polymeric covering 36 may be disposed within the plurality of anti-migration loops 30. In some embodiments, the polymeric covering 36 may be fixedly attached to the plurality of anti-migration loops 30. In some embodiments, the plurality of anti-migration loops 30 may be embedded within the polymeric covering 36. In some embodiments, each anti-migration loop of the plurality of anti-migration loops 30 may include a separate polymeric covering distinct from another anti-migration loop. In some embodiments, the polymeric covering 36 may include and/or may be formed as a pleated structure. In some embodiments, one polymeric covering may be secured to and/or fixedly attached to each row of the one or more rows of anti-migration loops 30 (e.g., one polymeric covering may be secured to each anti-migration loop within a given row of the one or more rows of anti-migration loops). Other configurations are also contemplated. In some embodiments, the polymeric covering 26 and the polymeric covering 36 may be formed from the same material. In some embodiments, the polymeric covering 26 and the polymeric covering 36 may be formed from different materials. Some suitable but non-limiting examples of materials that may be used to form the polymeric covering 36 are described below.

In some embodiments, each anti-migration loop of the plurality of anti-migration loops 30 may have an outer perimeter greater than an outer perimeter of each knitted loop of the plurality of knitted loops 20. In some embodiments, the plurality of anti-migration loops 30 may be larger than and/or may have a greater size than the plurality of knitted loops 20.

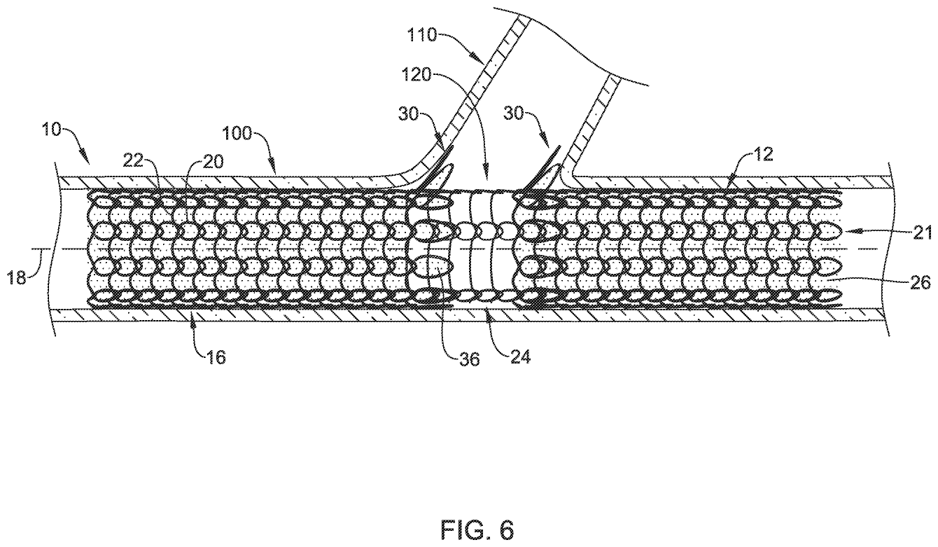

FIG. 6 illustrates selected aspects of and/or related to a method of placing the endoprosthesis 10 in a body lumen 100 having a branch lumen 110. In some embodiments, the method may include positioning the endoprosthesis 10 within the body lumen 100 adjacent the branch lumen 110. In the example shown in FIG. 6, the endoprosthesis 10 includes the plurality of anti-migration loops 30. In some embodiments, the method may include expanding the elongated tubular member 12 against a wall of the body lumen 100 such that the elongated tubular member 12 spans an opening 120 into the branch lumen 110. In some embodiments, the endoprosthesis 10 is positioned within the body lumen 100 adjacent the branch lumen 110 such that the at least one window 24 and/or the plurality of anti-migration loops 30 overlaps the opening 120 into the branch lumen 110. In some embodiments, the method may include shifting at least some of the plurality of anti-migration loops 30 from the first position toward the second position.

As described herein, in some embodiments, the plurality of anti-migration loops 30 may be biased toward the second position when unconstrained. In some embodiments, the plurality of anti-migration loops 30 may be self-biased toward the second position when unconstrained. As the elongated tubular member 12 expands against the wall of the body lumen 100, the plurality of anti-migration loops 30 may be constrained against the elongated tubular member 12 by the wall of the body lumen 100. The plurality of anti-migration loops 30 may not have enough outward biasing force to shift radially outward of the first position when the elongated tubular member 12 is expanded against and/or in contact with the wall of the body lumen 100. However, at any location where an opening in the body lumen 100 exists (e.g., the opening 120 into the branch lumen 110), the plurality of anti-migration loops 30 may shift away from the side wall of the elongated tubular member 12 and/or toward the second position due to a lack of resistance from the wall of the body lumen 100. Accordingly, the at least some of the plurality of anti-migration loops 30 shifted toward the second position may extend into the opening 120 of the branch lumen 110, as seen in FIG. 6.

In some embodiments, the endoprosthesis 10 may be positioned adjacent more than one branch lumen and/or opening in the wall of the body lumen 100. It is contemplated that the plurality of anti-migration loops 30 may shift from the first position toward the second position at any location where the plurality of anti-migration loops 30 overlaps an opening into the body lumen 100. Accordingly, while not expressly illustrated, in some embodiments, the plurality of anti-migration loops 30 may shift from the first position toward the second position at more than one location and/or may extend into more than one opening in the wall of the body lumen 100.

In at least some embodiments, after shifting the at least some of the plurality of anti-migration loops 30 from the first position toward the second position, all remaining loops of the plurality of anti-migration loops 30 may be trapped between the side wall of the elongated tubular member 12 and the wall of the body lumen 100 in the first position. Accordingly, the plurality of anti-migration loops 30 may be considered to react to the surrounding anatomy rather than forcing the surrounding anatomy to adapt to the plurality of anti-migration loops 30, which may have the potential to undesirably deform the body lumen 100, change fluid flow paths, damage or rupture the body lumen 100, etc.

Additionally, the endoprosthesis 10 may be used in body lumens devoid of openings and/or branch lumens. In such a use, the plurality of anti-migration loops 30 may remain in the first position after the endoprosthesis 10 and/or the elongated tubular member 12 is expanded toward and/or to the deployed configuration, and the endoprosthesis 10 may function as and/or similar to an endoprosthesis or stent lacking the plurality of anti-migration loops. Accordingly, the endoprosthesis 10 may scaffold the body lumen (e.g., a diseased body lumen or duct) as if treating with any other knitted stent. This functionality may provide flexibility in placement of the endoprosthesis 10 and thus permit fewer different endoprostheses or stents to be available, required, and/or used during a treatment procedure.

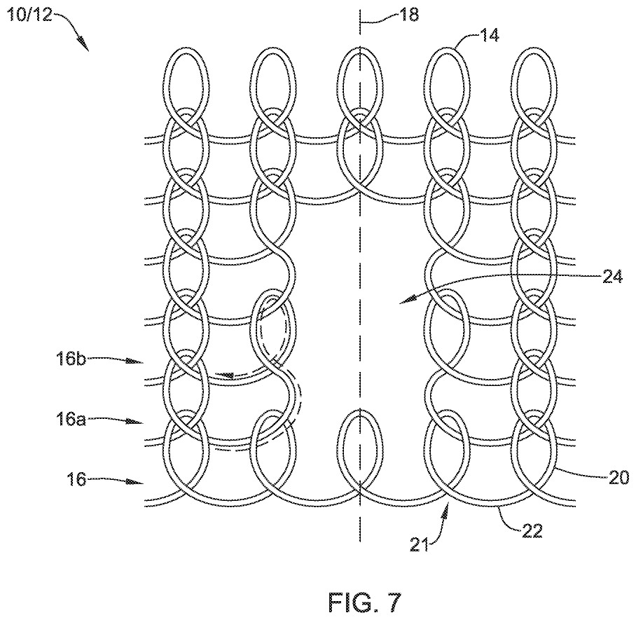

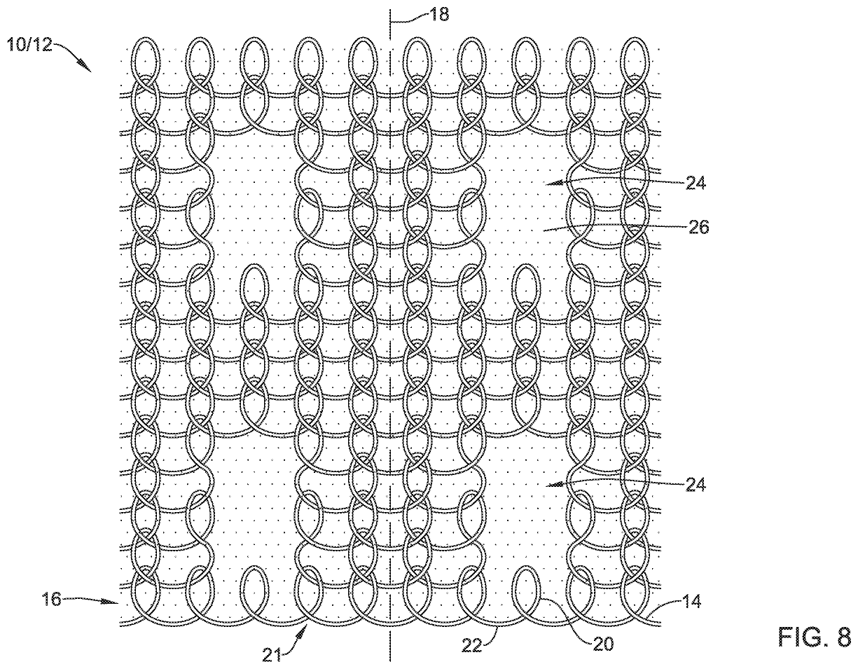

FIG. 7 illustrates selected aspects of a portion of the endoprosthesis 10 and/or the elongated tubular member 12. As discussed herein, in some embodiments, the elongated tubular member 12 may include at least one window 24 formed through the side wall of the elongated tubular member 12. The at least one window 24 may have a size equal to or greater than two knitted loops of the plurality of knitted loops 20. In some embodiments, the at least one window 24 may be devoid of intermediate rungs 22. In some embodiments, at the perimeter of the at least one window 24, the filament 14 may shift from one circumferential row 16A to an adjacent circumferential row 16B and reverse circumferential direction around the central longitudinal axis 18 of the elongated tubular member 12, as indicated by the dashed arrow in FIG. 7. This process and/or configuration may be repeated as desired to enlarge the at least one window 24 and/or to form additional windows. FIG. 8 illustrates one example configuration of the endoprosthesis 10 and/or the elongated tubular member 12 having a plurality of windows formed therein. As discussed above, in some embodiments, the filament 14 may extend continuously from the first end of the elongated tubular member 12 to the second end of the elongated tubular member 12. In some embodiments, the filament 14 may include and/or comprise one or more, and/or a plurality of, filaments.

In some embodiments, the elongated tubular member 12 may include the polymeric covering 26 disposed thereon, as seen in FIG. 8. In some embodiments, the polymeric covering 26 may be disposed between the first end of the elongated tubular member 12 and the second end of the elongated tubular member 12. In some embodiments, the polymeric covering 26 may extend between the first end of the elongated tubular member 12 and the second end of the elongated tubular member 12. In some embodiments, the polymeric covering 26 may extend from the first end of the elongated tubular member 12 to the second end of the elongated tubular member 12. In some embodiments, the at least one window 24 is devoid of the polymeric covering 26 (e.g., FIG. 7). In some embodiments, the at least one window 24 may include the polymeric covering 26 (e.g., FIG. 8). In some embodiments, the at least one window 24 may be partially covered by the polymeric covering 26. In some embodiments, the at least one window 24 may be completely covered by the polymeric covering 26.

In some embodiments, a method of making the endoprosthesis 10 comprising the elongated tubular member 12 may include knitting the filament 14 around the central longitudinal axis 18 to form the plurality of knitted loops 20 and intermediate rungs 22 extending between adjacent knitted loops in a first row of loops and rungs (e.g., circumferential row(s) 16). The method may include extending the filament 14 around the central longitudinal axis 18 to form a second row of loops and rungs (e.g., another circumferential row 16, circumferential row 16A, circumferential row 16B, etc.) interwoven with the first row of loops and rungs. The method may further include forming at least one window 24 through the side wall of the elongated tubular member 12 having a size equal to or greater than two knitted loops of the plurality of knitted loops 20 without cutting or severing the filament 14.

In some embodiments, forming the at least one window 24 through the side wall of the elongated tubular member 12 may include shifting the filament 14 from its current row of loops and rungs to a first subsequent row of loops and rungs, and forming the first subsequent row of loops and rungs interwoven with the current row of loops and rungs, as may be understood from FIGS. 7-8. In some embodiments, the current row of loops and rungs may be the first row of loops and rungs and the first subsequent row of loops and rungs may be the second row of loops and rungs. In some embodiments, the current row of loops and rungs may be the second row of loops and rungs and the first subsequent row of loops and rungs may be a third row of loops and rungs.

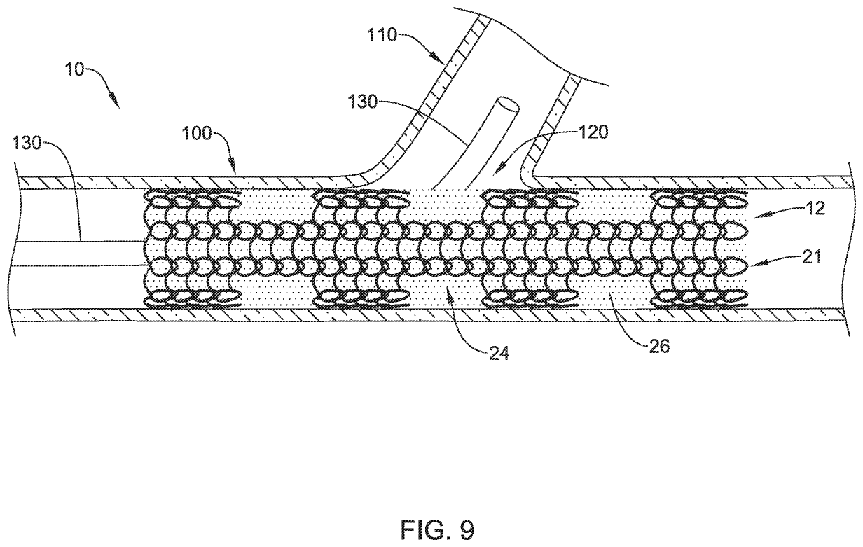

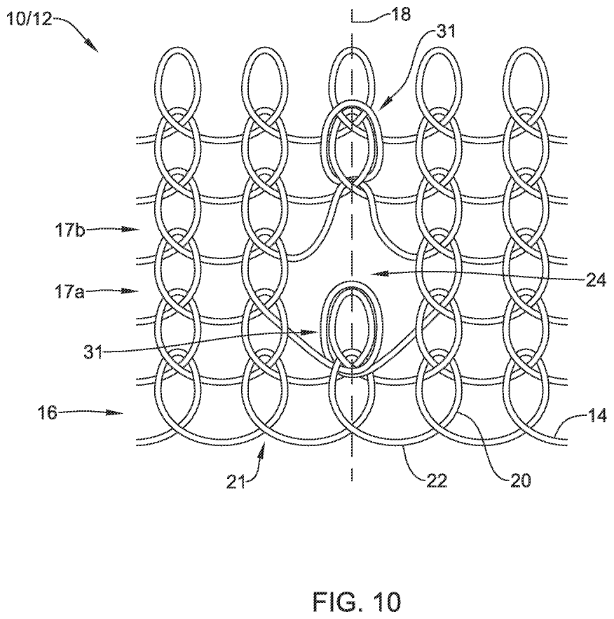

FIG. 9 illustrates one example use of and/or method of placing the endoprosthesis 10 shown in FIG. 8. In some embodiments, the method may include positioning the endoprosthesis 10 within the body lumen 100 adjacent the branch lumen 110. In the example shown in FIG. 9, the endoprosthesis 10 is devoid of the plurality of anti-migration loops 30 and the at least one window 24 includes the polymeric covering 26. In some embodiments, the method may include expanding the elongated tubular member 12 against the wall of the body lumen 100 such that the elongated tubular member 12 spans the opening 120 into the branch lumen 110. In some embodiments, the endoprosthesis 10 is positioned within the body lumen 100 adjacent the branch lumen 110 such that the at least one window 24 overlaps and/or spans the opening 120 into the branch lumen 110. In some embodiments, the at least one window 24 may function as an access port for treating the branch lumen 110. Alternatively, in some embodiments, the polymeric covering 26 may include small holes and/or may be pierced within the perimeter of the at least one window 24 to permit drainage between the branch lumen 110 and the body lumen 100 through the at least one window 24.

In some embodiments, a medical device 130 may be advanced into and/or through the body lumen 100 and into the endoprosthesis 10 and/or the elongated tubular member 12 within the body lumen 100. In some embodiments, the medical device 130 may be pushed and/or advanced through the polymeric covering 26 and/or the at least one window 24 into the branch lumen 110, as seen in FIG. 9. In some embodiments, a second medical device (e.g., a needle, a cutting device, etc.) may be used to pierce the polymeric covering 26 prior to advancing the medical device 130 through the polymeric covering 26 and/or the at least one window 24 into the branch lumen 110.

In some embodiments, the example configuration and/or method shown in FIG. 9 may use useful for Y-stenting procedures and/or when stenting a bifurcation to ensure that there is limited or no leakage between the two endoprostheses and/or stents. Additionally, in embodiments where the at least one window 24 includes a plurality of windows, the endoprosthesis 10 may provide flexibility in placement and/or the ability to treat more than one branch lumen via a single endoprosthesis. Other uses and/or benefits are also contemplated.

FIG. 10 illustrates selected aspects of a portion of the endoprosthesis 10 and/or the elongated tubular member 12. As discussed herein, in some embodiments, the elongated tubular member 12 may include at least one window 24 formed through the side wall of the elongated tubular member 12. The at least one window 24 may have a size equal to or greater than two knitted loops of the plurality of knitted loops 20. In some embodiments, the at least one window 24 may be devoid of intermediate rungs 22. In some embodiments, at the perimeter of the at least one window 24, the filament 14 may shift from one circumferential row to an adjacent circumferential row and the filament 14 may form at least one knitted loop 31 in the adjacent circumferential row that overlaps a knitted loop formed in the adjacent circumferential row before shifting back to the one circumferential row (e.g., the “original” circumferential row), as seen in FIG. 10. This process and/or configuration may be repeated for each window formed.

In one example, the filament 14 may first be shifted from a first circumferential row 17A to an adjacent circumferential row that is closer to the first end of the elongated tubular member 12, the filament 14 may form the at least one knitted loop 31, and the filament 14 may then shift back to the first circumferential row 17A. Next, the filament 14 may be shifted from a second circumferential row 17B immediately adjacent the first circumferential row 17A and offset toward the second end of the elongated tubular member 12 from the first circumferential row 17A to an adjacent circumferential row that is closer to the second end of the elongated tubular member 12, the filament 14 may form the at least one knitted loop 31, and the filament 14 may then shift back to the second circumferential row 17B.

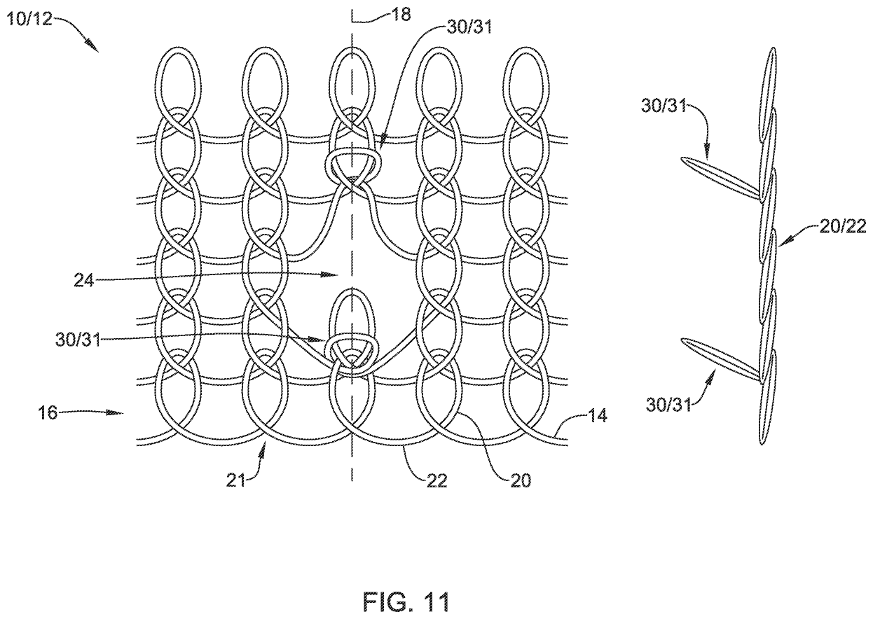

FIG. 11 illustrates an example configuration of the endoprosthesis 10 and/or the elongated tubular member 12 wherein the at least one knitted loop 31 forms the plurality of anti-migration loops 30. For the purpose of illustration only, FIG. 11 shows the plurality of anti-migration loops 30 in the second position, including in a schematic side view. The plurality of anti-migration loops 30 of FIG. 11 may function similarly to the plurality of anti-migration loops 30 shown in FIGS. 5-6. In some embodiments, the elongated tubular member 12 may include a plurality of windows and each window may have a plurality of anti-migration loops 30 disposed adjacent thereto. As discussed above, in some embodiments, the filament 14 may extend continuously from the first end of the elongated tubular member 12 to the second end of the elongated tubular member 12. In some embodiments, the filament 14 may include and/or comprise one or more, and/or a plurality of, filaments.

While not expressly illustrated, in some embodiments, the elongated tubular member 12 shown in FIGS. 10-11 may include the polymeric covering 26 disposed thereon. In some embodiments, the polymeric covering 26 may be disposed between the first end of the elongated tubular member 12 and the second end of the elongated tubular member 12. In some embodiments, the polymeric covering 26 may extend between the first end of the elongated tubular member 12 and the second end of the elongated tubular member 12. In some embodiments, the polymeric covering 26 may extend from the first end of the elongated tubular member 12 to the second end of the elongated tubular member 12. In some embodiments, the at least one window 24 is devoid of the polymeric covering 26. In some embodiments, the at least one window 24 may include the polymeric covering 26. In some embodiments, the at least one window 24 may be partially covered by the polymeric covering 26. In some embodiments, the at least one window 24 may be completely covered by the polymeric covering 26. In some embodiments, the plurality of anti-migration loops 30 may include the polymeric covering 36 as discussed herein.

In some embodiments, a method of making the endoprosthesis 10 comprising the elongated tubular member 12 may include knitting the filament 14 around the central longitudinal axis 18 to form the plurality of knitted loops 20 and intermediate rungs 22 extending between adjacent knitted loops in a first row of loops and rungs (e.g., circumferential row(s) 16, first circumferential row 17A, etc.). The method may include extending the filament 14 around the central longitudinal axis 18 to form a second row of loops and rungs (e.g., another circumferential row 16, first circumferential row 17A, second circumferential row 17B, etc.) interwoven with the first row of loops and rungs. The method may further include forming at least one window 24 through the side wall of the elongated tubular member 12 having a size equal to or greater than two knitted loops of the plurality of knitted loops 20 without cutting or severing the filament 14.

In some embodiments, forming at least one window 24 through the side wall of the elongated tubular member 12 may include shifting the filament 14 from its current row of loops and rungs (e.g., the first row of loops and rungs, the second row of loops and rungs, etc.) to an adjacent row of loops and rungs, forming at least one knitted loop 31 in the adjacent row of loops and rungs, and shifting the filament 14 back to the current row of loops and rungs to form at least one additional knitted loop of the plurality of knitted loops 20.

In some embodiments, the at least one knitted loop 31 may be interwoven with the elongated tubular member 12 and/or may be heat set to always remain in the first position. In some embodiments, the at least one knitted loop 31 may be heat set and/or self-biased to shift from the first position (e.g., FIG. 10) toward and/or to the second position (e.g., FIG. 11) when unconstrained. Other configurations, including combinations thereof, are also contemplated.

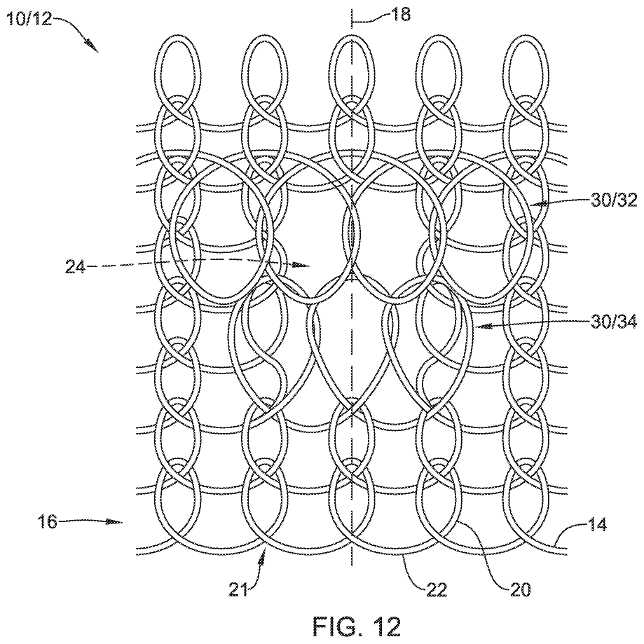

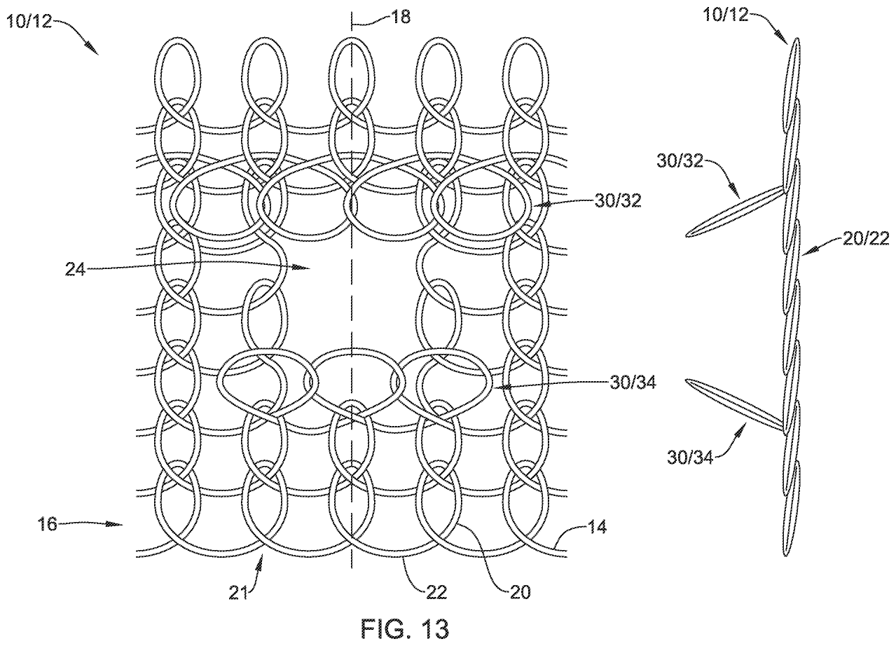

FIGS. 12-13 illustrate selected aspects of the endoprosthesis 10 and/or the elongated tubular member 12 including the at least one window 24 as described herein (e.g., FIG. 7). In some embodiments, the elongated tubular member 12 may include the plurality of anti-migration loops 30 disposed adjacent the at least one window 24. The plurality of anti-migration loops 30 may be configured to shift between the first position (e.g., FIG. 12) and the second position (e.g., FIG. 13). For the purpose of illustration only, FIG. 13 shows the plurality of anti-migration loops 30 in the second position, including in a schematic side view.

In some embodiments, in the first position, at least one first anti-migration loop 32 of the plurality of anti-migration loops 30 extends from intermediate rungs toward the first end of the elongated tubular member 12 and at least one second anti-migration loop 34 of the plurality of anti-migration loops 30 extends from intermediate rungs toward the second end of the elongated tubular member 12. In at least some embodiments, the at least one first anti-migration loop 32 and the at least one second anti-migration loop 34 may be self-biased toward the second position. In some embodiments, the at least one first anti-migration loop 32 and the at least one second anti-migration loop 34 may form a “barn door” configuration. In some embodiments, the at least one first anti-migration loop 32 and the at least one second anti-migration loop 34 may open and/or shift toward the second position in opposite directions (e.g., FIG. 13).

In at least some embodiments, the plurality of anti-migration loops 30, the at least one first anti-migration loop 32, and/or the at least one second anti-migration loop 34 may be formed from the filament 14. In some embodiments, the plurality of anti-migration loops 30, the at least one first anti-migration loop 32, and/or the at least one second anti-migration loop 34 may be disposed around the circumference of the elongated tubular member 12. In some embodiments, the plurality of anti-migration loops 30, the at least one first anti-migration loop 32, and/or the at least one second anti-migration loop 34 may be disposed completely around the circumference of the elongated tubular member 12 and/or around the entire circumference of the elongated tubular member 12.

In some embodiments, the plurality of anti-migration loops 30, the at least one first anti-migration loop 32, and/or the at least one second anti-migration loop 34 may be arranged in one or more rows of anti-migration loops. In some embodiments, the one or more rows of anti-migration loops may extend circumferentially around the elongated tubular member 12 and/or the central longitudinal axis 18. In some embodiments, the filament 14 may be doubled up around the circumference of the elongated tubular member 12 and/or within one or more circumferential rows 16 to form the plurality of anti-migration loops 30, the at least one first anti-migration loop 32, the at least one second anti-migration loop 34, and/or the one or more rows of anti-migration loops. In some embodiments, the filament 14 may be interwoven with an existing circumferential row 16 when forming the plurality of anti-migration loops 30, the at least one first anti-migration loop 32, the at least one second anti-migration loop 34, and/or the one or more rows of anti-migration loops. In some embodiments, the plurality of anti-migration loops 30, the at least one first anti-migration loop 32, the at least one second anti-migration loop 34, and/or the one or more rows of anti-migration loops may axially overlap an existing circumferential row 16 when forming the plurality of anti-migration loops 30, the at least one first anti-migration loop 32, the at least one second anti-migration loop 34, and/or the one or more rows of anti-migration loops. In some alternative embodiments, the plurality of anti-migration loops 30, the at least one first anti-migration loop 32, the at least one second anti-migration loop 34, and/or the one or more rows of anti-migration loops may be formed from a separate filament from the filament 14 forming the elongated tubular member 12. In those embodiments, the separate filament forming the plurality of anti-migration loops 30, the at least one first anti-migration loop 32, the at least one second anti-migration loop 34, and/or the one or more rows of anti-migration loops may be interwoven with the filament 14, the circumferential rows 16, and/or the elongated tubular member 12.

In some embodiments, the plurality of anti-migration loops 30, the at least one first anti-migration loop 32, and/or the at least one second anti-migration loop 34 may extend radially outward from the side wall and/or the central longitudinal axis 18 at an oblique angle relative to the side wall and/or the central longitudinal axis 18 in the second position and/or when unconstrained. In some embodiments, the plurality of anti-migration loops 30, the at least one first anti-migration loop 32, and/or the at least one second anti-migration loop 34 may extend radially outward from the side wall and/or the central longitudinal axis 18 at an acute angle relative to the side wall and/or the central longitudinal axis 18 in the second position and/or when unconstrained. In some embodiments, the plurality of anti-migration loops 30, the at least one first anti-migration loop 32, and/or the at least one second anti-migration loop 34 may extend radially outward from the side wall and/or the central longitudinal axis 18 at a right angle relative to the side wall and/or the central longitudinal axis 18 in the second position and/or when unconstrained. In some embodiments, the plurality of anti-migration loops 30, the at least one first anti-migration loop 32, and/or the at least one second anti-migration loop 34 may extend radially outward from the side wall and/or the central longitudinal axis 18 at an obtuse angle relative to the side wall and/or the central longitudinal axis 18 in the second position and/or when unconstrained.

In some embodiments, the plurality of anti-migration loops 30, the at least one first anti-migration loop 32, and/or the at least one second anti-migration loop 34 may extend radially outward from the side wall and/or the central longitudinal axis 18 at an angle of about 30 degrees, about 45 degrees, about 60 degrees, about 75 degrees, about 90 degrees, about 105 degrees, about 120 degrees, about 135 degrees, about 150 degrees, etc. relative to the side wall and/or the central longitudinal axis 18 in the second position and/or when unconstrained. In some embodiments, some anti-migration loops of the plurality of anti-migration loops 30, the at least one first anti-migration loop 32, and/or the at least one second anti-migration loop 34 may extend radially outward from the side wall and/or the central longitudinal axis 18 at different angles from each other relative to the side wall and/or the central longitudinal axis 18 in the second position and/or when unconstrained. Other configurations are also contemplated.

In some embodiments, the plurality of anti-migration loops 30, the at least one first anti-migration loop 32, and/or the at least one second anti-migration loop 34 may extend across the at least one window 24 in the first position. In some embodiments, the plurality of anti-migration loops 30, the at least one first anti-migration loop 32, and/or the at least one second anti-migration loop 34 may extend partially across the at least one window 24 in the first position. In some embodiments, the plurality of anti-migration loops 30, the at least one first anti-migration loop 32, and/or the at least one second anti-migration loop 34 may extend completely across the at least one window 24 in the first position. In some embodiments, the plurality of anti-migration loops 30, the at least one first anti-migration loop 32, and/or the at least one second anti-migration loop 34 may cover at least a portion of the at least one window 24 in the first position. In some embodiments, the plurality of anti-migration loops 30, the at least one first anti-migration loop 32, and/or the at least one second anti-migration loop 34 may cover at least 50% of the at least one window 24 in the first position. In some embodiments, the plurality of anti-migration loops 30, the at least one first anti-migration loop 32, and/or the at least one second anti-migration loop 34 may cover at least 60% of the at least one window 24 in the first position. In some embodiments, the plurality of anti-migration loops 30, the at least one first anti-migration loop 32, and/or the at least one second anti-migration loop 34 may cover at least 70% of the at least one window 24 in the first position. In some embodiments, the plurality of anti-migration loops 30 may cover at least 80% of the at least one window 24, the at least one first anti-migration loop 32, and/or the at least one second anti-migration loop 34 in the first position. In some embodiments, the plurality of anti-migration loops 30, the at least one first anti-migration loop 32, and/or the at least one second anti-migration loop 34 may cover at least 90% of the at least one window 24 in the first position. In some embodiments, the plurality of anti-migration loops 30, the at least one first anti-migration loop 32, and/or the at least one second anti-migration loop 34 may completely cover the at least one window 24 in the first position.

In some embodiments, the plurality of anti-migration loops 30, the at least one first anti-migration loop 32, and/or the at least one second anti-migration loop 34 may overlap each other in the first position. In some embodiments, the plurality of anti-migration loops 30, the at least one first anti-migration loop 32, and/or the at least one second anti-migration loop 34 may be positioned next to and/or adjacent each other without overlapping each other in the first position. In some embodiments, the at least one first anti-migration loop 32 overlaps the at least one second anti-migration loop 34 in the first position. In some embodiments, the at least one first anti-migration loop 32 axially overlaps the at least one second anti-migration loop 34 in the first position. the at least one first anti-migration loop 32 is circumferentially offset from and circumferentially overlaps the at least one second anti-migration loop 34 in the first position. Other configurations, including combinations thereof, are also contemplated.

In some embodiments, the plurality of anti-migration loops 30, the at least one first anti-migration loop 32, and/or the at least one second anti-migration loop 34 may include the polymeric covering 36 disposed thereon. In the interest of clarity, the polymeric covering 26 and the polymeric covering 36 are not shown in FIGS. 12-13. In some embodiments, the polymeric covering 36 may be disposed within the plurality of anti-migration loops 30, the at least one first anti-migration loop 32, and/or the at least one second anti-migration loop 34. In some embodiments, the polymeric covering 36 may be fixedly attached to the plurality of anti-migration loops 30, the at least one first anti-migration loop 32, and/or the at least one second anti-migration loop 34. In some embodiments, the plurality of anti-migration loops 30, the at least one first anti-migration loop 32, and/or the at least one second anti-migration loop 34 may be embedded within the polymeric covering 36. In some embodiments, each anti-migration loop of the plurality of anti-migration loops 30, the at least one first anti-migration loop 32, and/or the at least one second anti-migration loop 34 may include a separate polymeric covering distinct from another anti-migration loop. In some embodiments, the polymeric covering 36 may include and/or may be formed as a pleated structure. In some embodiments, one polymeric covering may be secured to and/or fixedly attached to each row of the one or more rows of anti-migration loops 30, the at least one first anti-migration loop 32, and/or the at least one second anti-migration loop 34 (e.g., one polymeric covering may be secured to each anti-migration loop within a given row of the one or more rows of anti-migration loops). Other configurations are also contemplated.

In some embodiments, each anti-migration loop of the plurality of anti-migration loops 30, the at least one first anti-migration loop 32, and/or the at least one second anti-migration loop 34 may have an outer perimeter greater than an outer perimeter of each knitted loop of the plurality of knitted loops 20. In some embodiments, the plurality of anti-migration loops 30, the at least one first anti-migration loop 32, and/or the at least one second anti-migration loop 34 may be larger than and/or may have a greater size than the plurality of knitted loops 20.

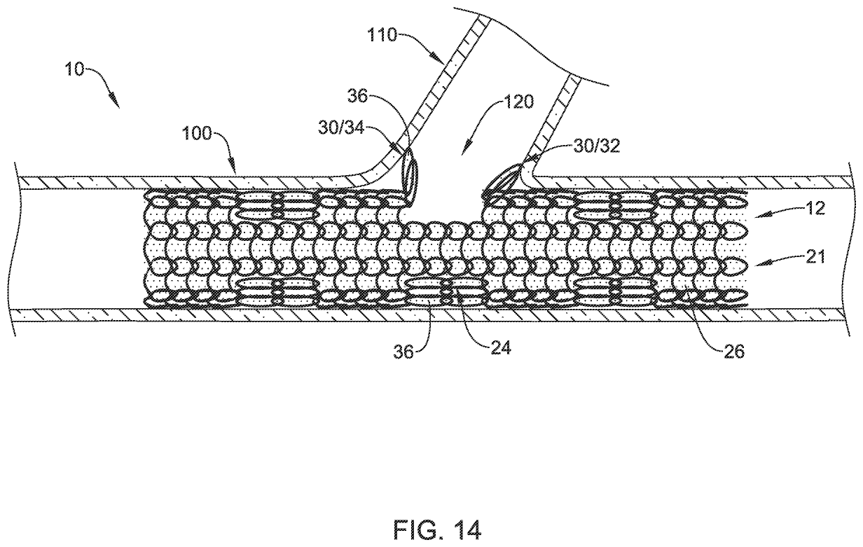

FIG. 14 illustrates selected aspects of and/or related to a method of placing the endoprosthesis 10 in the body lumen 100 having the branch lumen 110. In some embodiments, the method may include positioning the endoprosthesis 10 within the body lumen 100 adjacent the branch lumen 110. In the example shown in FIG. 14, the endoprosthesis 10 includes the plurality of anti-migration loops 30, the at least one first anti-migration loop 32, and/or the at least one second anti-migration loop 34. In some embodiments, the method may include expanding the elongated tubular member 12 against the wall of the body lumen 100 such that the elongated tubular member 12 spans the opening 120 into the branch lumen 110. In some embodiments, the endoprosthesis 10 is positioned within the body lumen 100 adjacent the branch lumen 110 such that the at least one window 24 and/or the plurality of anti-migration loops 30, the at least one first anti-migration loop 32, and/or the at least one second anti-migration loop 34 overlaps the opening 120 into the branch lumen 110. In some embodiments, the method may include shifting at least some of the plurality of anti-migration loops 30, the at least one first anti-migration loop 32, and/or the at least one second anti-migration loop 34 from the first position toward the second position.

As described herein, in some embodiments, the plurality of anti-migration loops 30, the at least one first anti-migration loop 32, and/or the at least one second anti-migration loop 34 may be biased toward the second position when unconstrained. In some embodiments, the plurality of anti-migration loops 30, the at least one first anti-migration loop 32, and/or the at least one second anti-migration loop 34 may be self-biased toward the second position when unconstrained. As the elongated tubular member 12 expands against the wall of the body lumen 100, the plurality of anti-migration loops 30, the at least one first anti-migration loop 32, and/or the at least one second anti-migration loop 34 may be constrained against the elongated tubular member 12 by the wall of the body lumen 100. The plurality of anti-migration loops 30, the at least one first anti-migration loop 32, and/or the at least one second anti-migration loop 34 may not have enough outward biasing force to shift radially outward of the first position when the elongated tubular member 12 is expanded against and/or in contact with the wall of the body lumen 100. However, at any location where an opening in the body lumen 100 exists (e.g., the opening 120 into the branch lumen 110), the plurality of anti-migration loops 30, the at least one first anti-migration loop 32, and/or the at least one second anti-migration loop 34 may shift away from the side wall of the elongated tubular member 12 and/or toward the second position due to a lack of resistance from the wall of the body lumen 100. Accordingly, the at least some of the plurality of anti-migration loops 30, the at least one first anti-migration loop 32, and/or the at least one second anti-migration loop 34 shifted toward the second position may extend into the opening 120 of the branch lumen 110, as seen in FIG. 14.

In some embodiments, the endoprosthesis 10 may be positioned adjacent more than one branch lumen and/or opening in the wall of the body lumen 100. It is contemplated that the plurality of anti-migration loops 30, the at least one first anti-migration loop 32, and/or the at least one second anti-migration loop 34 may shift from the first position toward the second position at any location where the plurality of anti-migration loops 30, the at least one first anti-migration loop 32, and/or the at least one second anti-migration loop 34 overlaps an opening into the body lumen 100. Accordingly, while not expressly illustrated, in some embodiments, the plurality of anti-migration loops 30, the at least one first anti-migration loop 32, and/or the at least one second anti-migration loop 34 may shift from the first position toward the second position at more than one location and/or may extend into more than one opening in the wall of the body lumen 100.

In at least some embodiments, after shifting the at least some of the plurality of anti-migration loops 30, the at least one first anti-migration loop 32, and/or the at least one second anti-migration loop 34 from the first position toward the second position, all remaining loops of the plurality of anti-migration loops 30, the at least one first anti-migration loop 32, and/or the at least one second anti-migration loop 34 may be trapped between the side wall of the elongated tubular member 12 and the wall of the body lumen 100 in the first position. Accordingly, the plurality of anti-migration loops 30, the at least one first anti-migration loop 32, and/or the at least one second anti-migration loop 34 may be considered to react to the surrounding anatomy rather than forcing the surrounding anatomy to adapt to the plurality of anti-migration loops 30, the at least one first anti-migration loop 32, and/or the at least one second anti-migration loop 34, which may have the potential to undesirably deform the body lumen 100, change fluid flow paths, damage or rupture the body lumen 100, etc.

Additionally, the endoprosthesis 10 may be used in body lumens devoid of openings and/or branch lumens. In such a use, the plurality of anti-migration loops 30, the at least one first anti-migration loop 32, and/or the at least one second anti-migration loop 34 may remain in the first position after the endoprosthesis 10 and/or the elongated tubular member 12 is expanded toward and/or to the deployed configuration, and the endoprosthesis 10 may function as and/or similar to an endoprosthesis or stent lacking the plurality of anti-migration loops. Accordingly, the endoprosthesis 10 may scaffold the body lumen (e.g., a diseased body lumen or duct) as if treating with any other knitted stent. This functionality may provide flexibility in placement of the endoprosthesis 10 and thus permit fewer different endoprostheses or stents to be available, required, and/or used during a treatment procedure.

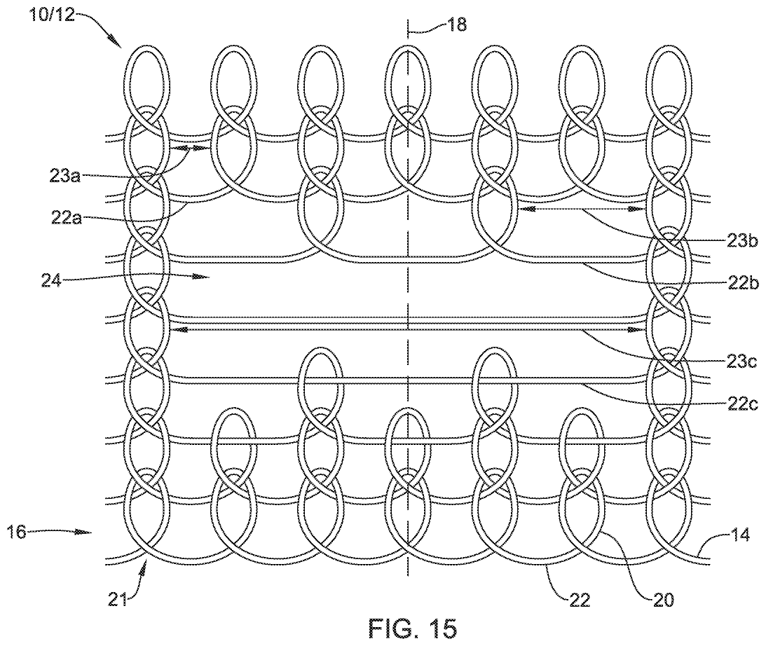

FIG. 15 illustrates selected aspects of an alternative configuration of the endoprosthesis 10 and/or the elongated tubular member 12 according to the disclosure. As described herein, the elongated tubular member 12 may comprise the filament 14 forming circumferential rows 16 disposed about the central longitudinal axis 18. The circumferential rows 16 may have a plurality of knitted loops 20 and intermediate rungs 22 extending between adjacent knitted loops of the plurality of knitted loops 20. In at least some embodiments, the filament 14 may extend continuously from the first end of the elongated tubular member 12 to the second end of the elongated tubular member 12. In some embodiments, the elongated tubular member 12 may include at least one window 24 formed through a side wall of the elongated tubular member 12. The at least one window 24 may have a size equal to or greater than two knitted loops of the plurality of knitted loops 20.

In some embodiments, the elongated tubular member 12 may have a density of knitted loops that is variable along its length, between its windows, and/or within its windows. In some embodiments, some circumferential rows 16 may have knitted loops that are spaced apart differently than other circumferential rows 16, and/or some circumferential rows 16 may have intermediate rungs of different length than other circumferential rows 16. In some embodiments, the density of knitted loops of the elongated tubular member 12 may be graduated and/or progressive along its length, between its windows, and/or within its windows.

For simplicity and clarity, the following discussion is made with reference to circumferential rows 16 that are considered “regular” and/or “consistent” in construction. It will be appreciated that in some embodiments, the circumferential rows 16 may be “irregular” and/or “inconsistent” in construction without deviating from the scope of the disclosure.

In one non-limiting example, a circumferential row 16 that is “dense” may have the greatest density of knitted loops within the elongated tubular member 12 (e.g., the highest quantity of knitted loops per circumferential row). In the non-limiting example shown in FIG. 15, a first circumferential row 16 that is “dense” may have intermediate rungs 22A having a first length and knitted loops that are spaced apart by a first distance 23A. In the non-limiting example shown in FIG. 15, a second circumferential row 16 that is “less dense” may have intermediate rungs 22B having a second length that is greater than the first length and knitted loops that are spaced apart by a second distance 23B that is greater than the first distance 23A. In the non-limiting example shown in FIG. 15, a third circumferential row 16 that is “least dense” may have intermediate rungs 22C having a third length that is greater than the second length and knitted loops that are spaced apart by a third distance 23C that is greater than the second distance 23B.

While varying density circumferential rows are generally illustrated in the non-limiting example of FIG. 15 as being arranged sequentially, this arrangement is not necessarily required. For example, in some embodiments, the second circumferential row 16 having intermediate rungs 22B having a second length that is greater than the first length and knitted loops that are spaced apart by a second distance 23B that is greater than the first distance 23A may be repeated and/or duplicated between instances of the first circumferential row 16 and the third circumferential row 16. Other arrangements and/or patterns of circumferential rows and/or windows are also contemplated.

The density of knitted loops being variable may permit the formation of larger windows and/or windows having a variable perimeter. In some embodiments, such windows may have advantageous drainage and/or flow characteristics while maintaining desired and/or necessary radial strength of the elongated tubular member 12.

The materials that can be used for the various components of the endoprosthesis and the various elements thereof disclosed herein may include those commonly associated with medical devices. For simplicity purposes, the following discussion refers to the device. However, this is not intended to limit the devices, components, and methods described herein, as the discussion may be applied to other elements, members, components, or devices disclosed herein, such as, but not limited to, the elongated tubular member, the filament, the knitted loops, the intermediate rungs, the anti-migration loops, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-clastic and/or super-clastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the device and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the device in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the device to achieve the same result.

In some embodiments, a degree of Magnetic Resonance Imaging (MRI) compatibility is imparted into the device and/or other elements disclosed herein. For example, the device and/or components or portions thereof may be made of a material that does not substantially distort the image and create substantial artifacts (e.g., gaps in the image). Certain ferromagnetic materials, for example, may not be suitable because they may create artifacts in an MRI image. The device or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-NR and the like), nitinol, and the like, and others.

In some embodiments, the device and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, cPTFE), a polyolefinic material such as a polyethylene, a polypropylene, polyester, polyurethane, and/or blends or combinations thereof.

In some embodiments, the device and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni—Co—Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the device and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An endoprosthesis, comprising:
an elongated tubular member comprising a filament forming circumferential rows disposed about a central longitudinal axis, the circumferential rows having a plurality of knitted loops and intermediate rungs extending between adjacent knitted loops;
wherein the filament extends continuously from a first end of the elongated tubular member to a second end of the elongated tubular member;
wherein the elongated tubular member includes at least one window formed through a side wall of the elongated tubular member having a size equal to or greater than two knitted loops of the plurality of knitted loops.

2. The endoprosthesis of claim 1, wherein intermediate rungs extend across the at least one window.

3. The endoprosthesis of claim 1, wherein the at least one window is devoid of intermediate rungs.

4. The endoprosthesis of claim 3, wherein at a perimeter of the at least one window the filament shifts from one circumferential row to an adjacent circumferential row and reverses circumferential direction around the central longitudinal axis of the elongated tubular member.

5. The endoprosthesis of claim 1, wherein the elongated tubular member includes a plurality of anti-migration loops disposed adjacent the at least one window, the plurality of anti-migration loops being configured to shift between a first position extending generally parallel to the side wall and a second position extending radially outward from the side wall;
wherein the plurality of anti-migration loops is self-biased toward the second position.

6. The endoprosthesis of claim 5, wherein in the first position, at least one first anti-migration loop of the plurality of anti-migration loops extends toward the first end of the elongated tubular member and at least one second anti-migration loop of the plurality of anti-migration loops extends toward the second end of the elongated tubular member.

7. The endoprosthesis of claim 6, wherein the at least one first anti-migration loop overlaps the at least one second anti-migration loop in the first position.

8. The endoprosthesis of claim 5, wherein the plurality of anti-migration loops extends across the at least one window in the first position.

9. The endoprosthesis of claim 8, wherein the plurality of anti-migration loops includes a polymeric covering disposed thereon.

10. The endoprosthesis of claim 5, wherein each anti-migration loop of the plurality of anti-migration loops has an outer perimeter greater than an outer perimeter of each knitted loop of the plurality of knitted loops.

11. The endoprosthesis of claim 1, wherein the elongated tubular member includes a polymeric covering disposed thereon between the first end and the second end.

12. The endoprosthesis of claim 11, wherein the at least one window is devoid of the polymeric covering.

13. A method of making an endoprosthesis comprising an elongated tubular member, the method comprising:
knitting a filament around a central longitudinal axis to form a plurality of knitted loops and intermediate rungs extending between adjacent knitted loops in a first row of loops and rungs;
extending the filament around the central longitudinal axis to form a second row of loops and rungs interwoven with the first row; and
forming at least one window through a side wall of the elongated tubular member having a size equal to or greater than two knitted loops of the plurality of knitted loops without cutting the filament.

14. The method of claim 13, wherein forming at least one window through the side wall of the elongated tubular member includes shifting the filament from its current row of loops and rungs to an adjacent row of loops and rungs, forming at least one knitted loop in the adjacent row of loops and rungs, and shifting the filament back to the current row of loops and rungs to form at least one additional knitted loop.

15. The method of claim 13, wherein forming at least one window through the side wall of the elongated tubular member includes shifting the filament from its current row of loops and rungs to a first subsequent row of loops and rungs, and forming the first subsequent row of loops and rungs interwoven with the current row of loops and rungs.

16. A method of placing an endoprosthesis in a body lumen having a branch lumen, comprising:
positioning an endoprosthesis within the body lumen adjacent the branch lumen, wherein the endoprosthesis comprises:
an elongated tubular member comprising a filament forming a plurality of knitted loops and intermediate rungs extending between adjacent knitted loops;
wherein the elongated tubular member includes at least one window formed through a side wall of the elongated tubular member having a size equal to or greater than two knitted loops of the plurality of knitted loops; and
a plurality of anti-migration loops disposed adjacent the at least one window, the plurality of anti-migration loops being configured to shift between a first position extending generally parallel to the side wall and a second position extending radially outward from the side wall;
expanding the elongated tubular member against a wall of the body lumen such that the elongated tubular member spans an opening into the branch lumen; and
shifting at least some of the plurality of anti-migration loops from the first position toward the second position;
wherein the at least some of the plurality of anti-migration loops shifted toward the second position extend into the opening of the branch lumen.

17. The method of claim 16, wherein after shifting the at least some of the plurality of anti-migration loops from the first position toward the second position, all remaining loops of the plurality of anti-migration loops are trapped between the side wall of the elongated tubular member and the wall of the body lumen in the first position.

18. The method of claim 16, wherein the plurality of anti-migration loops at least partially covers the at least one window in the first position.

19. The method of claim 16, wherein the endoprosthesis is positioned within the body lumen adjacent the branch lumen such that the at least one window overlaps the opening of the branch lumen.

20. The method of claim 16, wherein the plurality of anti-migration loops is formed from the filament forming the elongated tubular member.
